SEQUENCE LISTING (4 sequences)

SEQ ID NO: 1 - DNA, 1230 bp, Arabidopsis thaliana
ccacgcgtccgaaacactaaaagtagaagaaaaatggcgactcttaaggtttctgattctgttcctgctccttctgatgatgctgagcaattgagaaccgcttttgaaggatggggtacgaacgaggacttgatcatatcaatcttggctcacagaagtgctgaacagaggaaagtcatcaggcaagcataccacgaaacctacggcgaagaccttctcaagactcttgacaaggagctctctaacgatttcgagagagctatcttgttgtggactcttgaacccggtgagcgtgatgctttattggctaatgaagctacaaaaagatggacttcaagcaaccaagttcttatggaagttgcttgcacaaggacatcaacgcagctgcttcacgctaggcaagcttaccatgctcgctacaagaagtctcttgaagaggacgttgctcaccacactaccggtgacttcagaaagcttttggtttctcttgttacctcatacaggtacgaaggagatgaagtgaacatgacattggctaagcaagaagctaagctggtccatgagaaaatcaaggacaagcactacaatgatgaggatgttattagaatcttgtccacaagaagcaaagctcagatcaatgctacttttaaccgttaccaagatgatcatggcgaggaaattctcaagagtcttgaggaaggagatgatgatgacaagttccttgcacttttgaggtcaaccattcagtgcttgacaagaccagagctttactttgtcgatgttcttcgttcagcaatcaacaaaactggaactgatgaaggagcactcactagaattgtgaccacaagagctgagattgacttgaaggtcattggagaggagtaccagcgcaggaacagcattcctttggagaaagctattaccaaagacactcgtggagattacgagaagatgctcgtcgcacttctcggtgaagatgatgcttaatcaatcaatcctccacagagaaacataagctgctctacagcttctgttatctcttatctccctctctctctctttgatgagtttcaaatcgtttgattttgtttctacaaaaaccttgtttgtttctgttgtgtgttttgagttcctaaataatgcaaaagagagagacagagagaaccagtgtggtctcttaagttatatatatatgaagagcattggcctaaaaaaaaaaaaaaaaaaaaa

SEQ ID NO: 2 - protein, 317 aa, Arabidopsis thaliana
MATLKVSDSVPAPSDDAEQLRTAFEGWGTNEDLIISILAHRSAEQRKVIRQAYHETYGEDLLKTLDKELSNDFERAILLWTLEPGERDALLANEATKRWTSSNQVLMEVACTRTSTQLLHARQAYHARYKKSLEEDVAHHTTGDFRKLLVSLVTSYRYEGDEVNMTLAKQEAKLVHEKIKDKHYNDEDVIRILSTRSKAQINATFNRYQDDHGEEILKSLEEGDDDDKFLALLRSTIQCLTRPELYFVDVLRSAINKTGTDEGALTRIVTTRAEIDLKVIGEEYQRRNSIPLEKAITKDTRGDYEKMLVALLGEDDA

SEQ ID NO: 3 - DNA, 1093 bp, Arabidopsis thaliana
atggctcttcctctcgagctcgaaagcctcactgaagccatctcagctgggatgggaatgggagttgatgagaatgcattgataagcacactggggaaatcgcaaaaggaacatagaaaattgtttaggaaagcaagcaaaagtttctttgttgaagatgaggaaagagcttttgagaaatgtcatgatcacttcgtcagacacctcaagcttgagttctcccgcttcaatactgcggtggtgatgtgggcaatgcatccatgggagagagatgcaaggttggtgaagaaagctttgaagaaaggagaagaagcttacaacctcatcgttgaggtctcatgcacacgctctgctgaggatctcctcggtgcacgtaaagcttaccactctctcttcgaccaatcaatggaagaagacattgcctctcacgtccacggtcctcagcgcaagttgcttgtggggctcgtgagtgcttatagatacgaaggaaataaggtgaaggatgattctgccaaatccgatgctaagattctagccgaagcagtggcttcttcaggcgaagaagccgtggagaaggatgaggttgttaggattttgaccacaagaagcaaacttcatctccaacatctctacaaacactttaacgaaatcaaaggctctgatcttcttgggggtgtatctaagtcttctcttctcaatgaagcattgatttgtttgctcaaaccggctctgtatttcagcaagattttggatgcgtctctgaacaaagacgcagacaagactaccaagaaatggttgacaagagtgttcgttacaagagcagatcatagtgatgagatgaatgagatcaaagaagagtacaataacctttatggtgagactttggctcaaagaatccaagagaagataaaagggaactacagagatttcttgctcacacttctctccaaatccgattgatttcgtgttgagaaacctattaccaatacttttggttattgaagatttatgatttccctttttatggttttatgtttctaattcctaaatttgcgttttctcctaaaaaaaaaaaaaaaaaaaaaaaaaaaaa

SEQ ID NO: 4 - protein, 319 aa, Arabidopsis thaliana
MALPLELESLTEAISAGMGMGVDENALISTLGKSQKEHRKLFRKASKSFFVEDEERAFEKCHDHFVRHLKLEFSRFNTAVVMWAMHPWERDARLVKKALKKGEEAYNLIVEVSCTRSAEDLLGARKAYHSLFDQSMEEDIASHVHGPQRKLLVGLVSAYRYEGNKVKDDSAKSDAKILAEAVASSGEEAVEKDEVVRILTTRSKLHLQHLYKHFNEIKGSDLLGGVSKSSLLNEALICLLKPALYFSKILDASLNKDADKTTKKWLTRVFVTRADHSDEMNEIKEEYNNLYGETLAQRIQEKIKGNYRDFLLTLLSKSD

(19) United States
(12) Patent Application Publication
(10) Pub. No.: US 2005/0089872 A1
(43) Pub. Date: Apr. 28, 2005

(54) Nucleic acid molecules encoding annexins from plants

(76) Inventors: Ohkmae Kim, Kwangju (KR); Sumin Lee, Kwangju (KR); Ae Ran Park, Kwangju (KR)

Correspondence Address: JACOBSON HOLMAN PLLC, 400 SEVENTH STREET N.W., SUITE 600, WASHINGTON, DC, 20004, US

(21) Appl. No.: 10/690,564

(22) Filed: Oct. 23, 2003

(51) Int. Cl. C12Q 1/68; C07H 21/04; A01H 1/00; C12N 15/82; C07K 14/415; C12N 5/04

(52) U.S. Cl. 435/6; 435/69.1; 435/419; 435/468; 530/370; 536/23.6; 800/289

(57) ABSTRACT

The present invention provides nucleic acid molecules encoding annexin 1 (ANX1) and annexin 4 (ANX4) that belong to a multigene family of calcium-dependent membrane binding proteins. ANX1 and ANX4 are inducible by abscisic acid (ABA), salt and other various stress treatments. The anx1 and anx4 mutant plants are characterized by their hypersensitivity to salt and ABA during seed germination and early seedling growth. The invention can be utilized to generate transgenic plants that are tolerant to environmental stress.

### FIELD OF THE INVENTION

This invention relates to the field of stress resistance in plants. In particular, the invention provides novel genes from plants, which encode calcium-dependent membrane binding proteins, ANX1 and ANX4, involved in osmotic stress and ABA signaling to protect plants from environmental stress.

### BACKGROUND OF THE INVENTION

Soil salinity is one of the most significant abiotic stress especially for crop plants, leading to reduction of productivity. Plants have the capacity to adapt to ionic and osmotic stress caused by salinity. Salt stress causes accumulation of excess toxic Na⁺, along with deficiency of K⁺ and turgor changes in the cytosol, leading to the disruption of ionic and osmotic homeostasis, respectively. Salt-induced ionic stress is clearly distinct from other stress, while osmotic stress is generally induced by salt, cold and drought.

The Salt Overly Sensitive (SOS) pathway for ionic stress signaling has been elucidated through genetic analyses (Wu et al., 1996; Liu and Zhu, 1997; Zhu et al., 1998). SOS3, a Ca²⁺-binding protein, senses the Ca²⁺ change elicited by salt stress (Quintero et al., 2002). The SOS3—SOS2 kinase complex regulates the expression and transport activity of ion transporters such as SOS1, a plasma membrane Na⁺/H⁺ exchanger, eventually removing Na⁺ from the cytosol (Qiu et al., 2002).

Evidence has been presented that osmotic stress activates signaling pathways distinct from the SOS pathway, with the identification of several protein kinases activated by osmotic stress (Zhu, 2002). Mitogen-activated protein kinases (MAPKs) are activated by hyperosmotic stress (Xiong et al., 2002). In Arabidopsis, at least three MAPKs are activated by salt and other stress (Ichimura et al., 2000; Drolliard et al., 2002). Ca²⁺-dependent protein kinases (CDPKs) have been implicated in osmotic stress response in association with Ca²⁺ signaling (Romeis et al., 2001).

The plant hormone, abscisic acid (ABA), plays a critical role in stress responses (Giraudat et al., 1994). Osmotic and cold stress induce increased levels of ABA (Zeevaart and Creelman, 1998). Osmotic stress elicited by water deficit or high salt alters the expression of numerous genes (Skriver and Mundy, 1990). Some of these genes are also induced by ABA, suggesting that the hormone mediates stress signaling (Skriver and Mundy, 1990). Molecular studies have led to the identification of cis- and trans-acting elements controlling stress-inducible genes involved in ABA-dependent and independent pathways (Yamaguchi-Shinozaki and Shinozaki, 1994; Grill and Himmelbach, 1998; Shinozaki and Yamaguchi-Shinozaki, 2000).

ABA, cold, drought and salt stress trigger elevations in the cytosolic Ca²⁺ level in plant cells (Knight et al., 1996; Knight and Knight, 2001). As a second messenger, Ca²⁺ activates signaling pathways and therefore influences multiple aspects of cellular functions (Knight et al., 1996; Knight et al., 1997; Trewavas, 1999). Ca²⁺-binding proteins serve as transducers of the Ca²⁺ signal. Ca²⁺-binding proteins have been identified in plants, such as calmodulin (Zielinski, 1998; Luan et al., 2002), CDPKs (Harmon et al., 2000), calcineurin B-like proteins (CBLs) (Luan et al., 2002) and SOS3 (Liu and Zhu, 1998). A number of these proteins are involved in ABA and abiotic stress responses (Sheen, 1996; Sajio et al., 2000; Townely and Knight, 2002).

Despite considerable progress in understanding stress signal transduction, the mechanisms of stress response largely remain unknown. The identification of novel signaling components will contribute to the clarification of stress signaling. Following the completion of genome sequencing in Arabidopsis, the identification of stress-responsive proteins is currently feasible with proteomics. In this study, the microsomal proteome from Arabidopsis roots was isolated and analyzed using two-dimensional (2D) gel electrophoresis and matrix-assisted laser desorption/ionization-time of flight mass spectrometry (MALDI-TOF MS). In an attempt to identify the membrane proteins involved in salt stress, we evaluated salt-induced changes in the microsomal proteome and identified Ca²⁺-dependent membrane binding proteins, designated annexins, as the signaling components of stress response. 2D gel analyses combined with Western blotting revealed that levels of annexin 1 (ANX1) significantly increase in the microsome in a Ca²⁺-dependent manner in response to osmotic stress. The anx1 and anx4 mutant plants were hypersensitive to salt and ABA during seed germination and early seedling growth. Based on these findings, we propose that ANXs comprise a novel class of Ca²⁺-binding proteins that play essential roles in ABA-mediated stress response in plants.

### SUMMARY OF THE INVENTION

The present invention relates to nucleic acid molecules as given in SEQ ID NO:1 and SEQ ID NO: 3. Such nucleic acid molecules preferentially encode ANX1 and ANX4 proteins with the amino acid sequences as given in SEQ ID NO: 2 and SEQ ID NO: 4, or biologically active fragments of such proteins.

The present invention relates to polypeptides or biologically active fragments of such polypeptides encoded by said nucleic acid molecules. ANX1 and ANX4 proteins coded by said nucleic acid molecules belong to a multigene family of calcium-dependent membrane binding proteins and play essential roles in ABA-mediated stress response to protect plants from environmental stress. Further, the invention describes polypeptides that are identified as the salt-responsive microsomal proteins and strongly expressed in root tissues. The polypeptides coded by the above-described nucleic acid molecules significantly increase in the microsome in a Ca²⁺-dependent manner in response to ABA, salt and other osmotic stress.

The invention also relates to vectors, expression cassettes and plasmids used in genetic engineering that contain the nucleic acid molecules as described above according to the invention.

In one aspect, the present invention relates to transgenic plant cells and plants containing said nucleic acid molecules. The provision of the nucleic acid molecules according to the present invention offers the potential to generate transgenic plants with the increased resistance against environmental stress. This is based on the findings that the anx1 and anx4 mutant plants are hypersensitive to salt and ABA during seed germination and early seedling growth.

With the present invention, it is possible to engineer plant growth, in regard to the improvement of resistance to environmental damages, by introducing the ANX1 and ANX4 into economically important crop plants in an organ-specific manner.

Therefore, the present invention provides:

Nucleic acid molecules encoding ANX1 and ANX4 that are a multigene family of calcium-dependent membrane binding proteins, and involved in osmotic stress and ABA signaling to protect plants from environmental stress, comprising nucleotide sequences as given in SEQ ID NO: 1 and SEQ ID NO: 3.

### BRIEF DESCRIPTION OF THE FIGURES

FIG. 1. 2D gel electrophoresis analysis of root microsomal proteins. (A) Microsomal proteins resolved in the range of pH 4-7. (B) NaCl-responsive microsomal proteins. Salt-responsive changes in protein expression were analyzed in gels prepared with the microsomal proteins from seedlings left untreated (left) or treated with 250 mM NaCl (right) for 2 h.

FIG. 2. Expression of ANX1 in tissues. Crude extracts from various tissues were separated by SDS gel electrophoresis and subjected to Coomassie staining (right) and Western analysis with an anti-ANX1 antibody (left).

FIG. 3. Expression of ANX1 in response to abiotic stress. Two-week-old seedlings grown in MS liquid media were incubated with the indicated treatments for 2 h. Microsomal proteins prepared from root tissue were subjected to 2D gel electrophoresis and Western blotting with an anti-ANX1 antibody. (A) NaCl dose response of microsomal ANX1 proteins. (B) Treatment with 20% PEG, 0.25 M mannitol and 100 μM ABA.

FIG. 4. Salt and calcium response of ANX1 proteins. Proteins in microsomal (Microsome) and cytosolic (Cytosol) fractions and the total protein extract (Total) prepared from roots of two-week-old seedlings were subjected to 2D gel electrophoresis and Western blotting with an anti-ANX1 antibody. (A) ANX1 localization in response to NaCl. (B) In vitro ANX1 localization in response to Ca²⁺. The total protein extract was treated with either 2 mM CaCl₂ or 2 mM EGTA for 15 min before fractionation into the microsome and cytosol. (C) In vivo ANX1 localization in response to Ca²⁺. Plants left untreated (−) or treated (+) with 10 mM EGTA for 30 min were further incubated with (+) or without (−) 250 mM NaCl for 2 h. (D) Northern analysis of ANX1 expression in response to NaCl.

FIG. 5. T-DNA insertion mutants of ANX genes. (A) Scheme of ANX genes. The arrows indicate the positions of the T-DNA insertions (triangle) within the ANX1, ANX2 and ANX4 alleles. (B) RNA analysis of ANX gene expression in wild type, anx1, anx2 and anx4 plants. (C) Western analysis of ANX1 expression in wild type and anx1 plants.

FIG. 6. Sensitivity of anx mutant plants to NaCl. Seeds of wild type (WT) and anx mutants were plated on MS media alone or supplemented with various concentrations of NaCl. The percentage of germinated seeds was determined at various times. (A) Germination rates of wild type and anx mutants on MS media. (B) Germination rates of wild type and anx mutants on MS media containing 50 mM NaCl. (C) Germination rates of wild type and anx mutants on MS media containing 75 mM NaCl. (D) NaCl dose response of germination. (E) The effect of NaCl on germination. Seeds of wild type and anx mutants were plated on MS media alone or supplemented with 75 mM NaCl and allowed to germinate for 4 days.

FIG. 7. Sensitivity of anx1 and anx4 mutant plants to osmotic stress. Seeds of wild type and anx mutants were plated on MS media alone or supplemented with various concentrations of mannitol (A), KCl (B), LiCl (C) or CsCl (D). The percentage of germinated seeds was determined at 4 days after plating.

FIG. 8. Sensitivity of anx1 and anx4 mutant plants to ABA. (A) Germination rates of wild type and anx mutants on MS media containing 0.5 μM ABA. The percentage of germinated seeds was determined at the indicated times. (B) ABA dose response of germination. (C) The effect of ABA on germination. Seeds of wild type, abi1 and anx mutants were plated on MS media containing 0.25 and 0.5 μM ABA and allowed to germinate for 4 days. (D) The effect of ABA on early seedling growth. Seeds of wild type and anx mutants were germinated and grown on MS media containing 0.25 μM ABA for 7 days.

### DETAILED DESCRIPTION OF THE INVENTION

Membrane proteins play important roles in various cellular processes, modulating diverse signaling pathways. Many signals are initially perceived and transduced through active molecules located in the membrane, which regulate cell-cell interactions and responses to the environment. Therefore, we targeted the microsomal proteome containing active proteins such as receptors, channels and membrane-associated signaling molecules for analysis. In this study, proteomic analyses led to the identification of the ANX1 protein. Levels of ANX1 increased upon NaCl treatment in the root microsomal proteome from Arabidopsis. Annexins are a family of Ca²⁺-dependent membrane binding proteins that exist in nearly all species, from fungi to human (Gerke and Moss, 2002). Annexins have been extensively studied in animal cells. These proteins are multifunctional and play important roles in various cellular processes, including membrane trafficking and organization, regulation of ion channel activity, phospholipid metabolism, inflammatory response and mitotic signaling (Raynal and Pollard, 1994).

Thus the present invention provides nucleic acid molecules encoding ANX1 and ANX4, or biologically active fragments of such proteins that are a multigene family of calcium-dependent membrane binding proteins, and involved in osmotic stress and ABA signaling in plants. The nucleic acid molecules preferentially encode proteins with the amino acid sequences as given in SEQ ID NO: 2 and SEQ ID NO: 4, or fragments thereof that possess the activities of the above-mentioned ANX1 and ANX4. Such nucleic acid molecules as given in SEQ ID NO: 1 and SEQ ID NO: 3 are preferred. Furthermore, the invention relates to the plasmids and expression cassettes comprising nucleic acid molecules containing the nucleotide sequences as given in SEQ ID: 1 and SEQ ID: 3 for functional expression in prokaryotic and eukaryotic cells. The nucleic acid molecules can be isolated as a full-size cDNA clone by various conventional methods, such as reverse transcriptase-mediated PCR (RT-PCR) using mRNA or by the screening of a cDNA library using partial-size cDNA clones as probes, well-known techniques to the art. For the RT-PCR method, the poly(A)+ mRNA can be first converted into a primary cDNA using the reverse transcriptase and the oligo(dT)¹⁶⁻¹⁸ as the primer. An uninterrupted double stranded cDNA can then be synthesized by PCR using a pair of specific primers.

The present invention also relates to nucleic acid molecules that hybridize under high stringent conditions to nucleic acid molecules as given in SEQ ID: 1 and SEQ ID: 3. The term “hybridize under high stringent conditions” means that such nucleic acid molecules hybridize through complementary base pairing under conventional hybridization conditions, as described in Sambrook et al., (Molecular Cloning: A Laboratory Manual, 2^{nd} Ed., Cold Spring Harbor Laboratory, Cold Spring Harbor, N.Y., 1989). Nucleic acid molecules hybridizing with the above nucleic acid molecules include in general those from any plants, preferentially from plants of interest in agriculture, forestry and horticulture, such as rice, barley, wheat, oilseed rape, potato, tomato, cabbage, lettuce, spinach, melon, watermelon, green onion, radish, cauliflower, sugar cane, cucumber and sugar beet. Woody plants are also preferred sources. To isolate nucleic acid molecules that hybridize to the nucleic acid molecules as given in SEQ ID: 1 and SEQ ID: 3, a cDNA or a genomic DNA library is screened using the above-described nucleic acid molecules as probes, a molecular biological technique well known to the art.

According to the present invention, the term “degenerate” means that the nucleotide sequences of nucleic acid molecules are different from the above-described nucleic acid molecules in one or more base positions and highly homologous to said nucleic acid molecules. “Homologous” indicates the amino acid sequence identity of at least 70%, particularly 80% higher. The term also includes derivatives of the nucleic acid molecules as described above by insertions, deletions, base substitutions and recombinations. The “homologous” also describes that the nucleic acid molecules or the polypeptides coded by said nucleic acid molecules are structurally and functionally equivalent.

Furthermore, the present invention relates to polypeptides or biologically active fragments of such polypeptides coded by said nucleic acid molecules for the use in the protein analysis and biochemical assays. One efficient way to get such polypeptides is to use the recombinant expression systems. To do this, the nucleic acid molecule is first inserted into an expression vector containing regulatory elements required for efficient expression of the polypeptide coded by said nucleic acid molecule, such as promoters, terminators and polyadenylation signals. The expression cassettes are then transfected into appropriate host cells. The host cells can be prokaryotic or eukaryotic. For efficient isolation of the expressed polypeptide from the host cell culture, affinity tags are attached to the polypeptide. The tags can be easily removed from the fusion proteins after isolation by enzymatic or biochemical methods, a recently well-established skill to the art.

The protein activity of ANX1 and ANX4 coded by said nucleic acid molecules can be assayed based on the property that the proteins bind to membrane or phospholipids in a calcium-dependent manner. The polypeptides coded by the above-described nucleic acid molecules share common structural and functional properties with annexins.

The present invention also relates to vectors, expression cassettes and plasmids used in genetic engineering that contain the nucleic acid molecules as described above according to the invention.

The present invention can be utilized to generate transgenic plant cells and plants containing said nucleic acid molecules and to the processes and methods for the elucidation of other proteins and molecular events involved in ABA-mediated stress signaling. The provision of the nucleic acid molecules according to the present invention offers the potential to generate transgenic plants with a reduced or increased resistance to various stress. Technical procedures for the generation of transgenic plant cells and plants are well known to the art.

With recent technical advances in plant tissue culture and manipulation of genetic materials, it is at present a routine procedure to introduce a new desired gene into economically important plants to improve plant productivity and quality. The nucleic acid molecules in the present invention can be potential targets for such purpose. For example, they can be utilized to engineer plant resistance to environmental stress and damages. The desired plants for the embodiment of the present invention include any of valuable plants in agriculture, forestry and horticulture, such as rice, corn, sugar cane, turf grass, melon, watermelon, cucumber, pepper and popular tree. Plants in horticulture whose quality can be improved by engineering stress resistance are also good target plants for the embodiment of the present invention;
Therefore, the present invention provides:

Nucleic acid molecules encoding ANX1 and ANX4 that are a multigene family of calcium-dependent membrane binding proteins, and involved in osmotic stress and ABA signaling to protect plants from environmental stress, comprising nucleotide sequences as given in SEQ ID NO: 1 and SEQ ID NO: 3.

### EXAMPLES

Plant Materials and Growth Conditions

Arabidopsis thaliana ecotype Columbia (Col-0) was grown in a growth room under long-day conditions (16 h/8 h, light/dark cycle). T-DNA insertion mutants, anx1 (SALK_{—}015426), anx2-1 (SALK_{—}054223), anx2-2 (SALK_{—}054238), anx4-1 (SALK_{—}109725), anx4-2 (SALK_{—}039476) and anx4-3 (SALK_{—}073121), were obtained from the Arabidopsis Biological Resources Center (ABRC) (Columbus, Ohio, USA). For plant materials, plants were either grown in soil for 3 weeks or in Murashige and Skoog (1962) (MS)-sucrose (2%) liquid medium for 2 weeks.

Germination Test

For seed germination analysis, sterilized seeds were plated on MS-sucrose (2%) agar medium. Various concentrations of NaCl, KCl, ABA, mannitol, LiCl and CsCl were added, as described in the Results section. Germination (emergence of radicles) was scored daily for days. Three replicate plates were used for each treatment to ensure reproducibility of data.

Preparation of Microsomal and Cytosolic Proteins

Arabidopsis seedlings were grown for 2 weeks in liquid MS medium with continuous shaking and treated with various concentrations of NaCl, ABA, PEG and mannitol for the indicated times. Roots were harvested, immediately frozen and ground in liquid nitrogen. The ground root powder was incubated in extraction buffer (50 mM Tris, pH 8.0, 2 mM EDTA, 2 mM DTT, 0.25 M sucrose, and protease inhibitor cocktail) and subjected to centrifugation at 8,000 g for 15 min. The supernatant (total protein extract) was repeatedly centrifuged at 100,000 g for 1 h. Following centrifugation, the supernatant (cytosolic fraction) was recovered, the pellet (microsomal fraction) re-washed with extraction buffer by further centrifuging at 100,000 g for 1 h and dissolved in an appropriate volume of extraction buffer. Isolated cytosolic and microsomal fractions were divided into aliquots and either used immediately or frozen at −80° C. For Western analysis of 2D-gels, 80 μg of microsomal proteins, and 40 μg of cytosolic and total proteins were used.

Two-Dimensional Gel Electrophoresis

To remove lipids that interfere with isoelectric focusing (IEF), 200 μg of microsomal proteins in 200 μL were extracted with the same volume of TE (10 mM Tris, pH 8.0, and 1 mM EDTA)-saturated phenol. After centrifugation at 12,000 g for 10 min, the upper aqueous phase was removed without disturbing the interface. The lower phase, including interface, was re-extracted with 2 volumes of cold phenol-saturated TE buffer. After centrifugation, the upper phase was removed and proteins were precipitated with 5 volumes of 0.1 M ammonium acetate in methanol. Precipitated proteins were washed three times with 0.1 M ammonium acetate in methanol and once with 80% acetone.

The pellet was dried and dissolved in IEF sample buffer (7 M urea, 2 M thiourea, 0.05% dodecylmaltoside, 4% CHAPS, 20 mM Tris, 20 mM DTT, 0.5% IPG buffer, and 0.001% bromophenol blue). Immobiline™ DryStrips (pH 4-7) (Amersham Biosciences, Uppsala, Sweden) were rehydrated with proteins and focused on the IPGphor system (Amersham Biosciences). Strips were transferred to equilibration buffer (50 mM Tris, pH 8.8, 6 M urea, 30% glycerol, 2% SDS, and 20 mM tributylphosphine) and incubated for 15 min. Equilibrated strips were placed on top of vertical polyacrylaminde gels and overlaid with 0.5% agarose in SDS running buffer. Following electrophoresis, proteins were visualized by silver staining.

Sample Preparation for MALDI-TOF MS

Peptide samples were prepared, as described previously (Jensen et al., 1999). Protein spots were excised from the gel, reduced, alkylated and digested with trypsin. Tryptic-digested peptides were recovered through a series of extraction steps. Extraction with 25 mM ammonium bicarbonate and acetonitrile was followed by second extraction with 5% trifluoroacetic acid (TFA) and acetonitrile. Extracts were pooled and lyophilized in a vacuum lyophilizer. Lyophilized tryptic peptides were redissolved in solution containing water, acetonitrile and TFA (93:5:2), and bath-sonicated for 5 min. The peptide extract was prepared using the “solution-phase nitrocellulose” method (Landry et al, 2000).

MALDI-TOF MS and Database Searching

Peptide masses were measured on a MALDI-TOF MS (Voyager-DE STR, Perceptive Biosystems, NA, USA). Peptide mass fingerprint data were matched to the NCBI nonredundant database entries, using the MS-Fit program available at the UCSF server (http://prospector.ucsf.edulucsfhtml4.0/msfit.htm). The following search parameters were applied. Mass tolerance was set to 50 ppm and one incomplete cleavage was allowed. Acetylation of the N-terminus, alkylation of cysteine by carbamidomethylation, oxidation of methionine, and pyroGlu formation of N-terminal Gln were set as possible modifications.

Antibody Generation and Protein Analysis

A polyclonal antibody was raised in rat to an ANX1-specific peptide (amino acids 204-215, NRYQDDHGEEIL). Proteins were separated on 12% SDS-polyacrylamide gels, transferred onto nitrocellulose membranes and incubated with the anti-ANX1 antibody overnight at 4° C. Antibody-bound proteins were detected following incubation with secondary antibody conjugated to horseradish peroxidase, using the ECL system (Amersham Biosciences).

RNA Analysis

RNA was isolated using the TR1 reagent (MRC, Ohio, USA), according to the manufacturer's instructions. For RNA gel blot analysis, 30 μg of total RNA was fractionated on a 1.2% formaldehyde-agarose gel, transferred to a nylon membrane (Hybond N+, Amersham Biosciences) and fixed using the UV crosslinker (Stratagene, USA). Loading of equal amounts of RNA was confirmed by ethidium bromide staining. Hybridization was performed in Rapid-Hyb buffer (Amersham Biosciences) for 16-24 h at 65° C. Following hybridization, membranes were serially washed in 2×SSC/0.1% SDS, 1×SSC/0.1% SDS and finally 0.1% SSC/0.1% SDS. RNA bands were visualized by autoradiography. Reverse transcriptase-polymerase chain reaction (RT-PCR) was performed with 0.4 and 0.1 μg of total RNA for detection of ANX4 and Actin, respectively, using the Access RT-PCR system (Promega, Wis., USA).

Results

Proteomic Identification of Salt Stress-Responsive Microsomal Proteins in Arabidopsis

To identify salt stress-regulated microsomal proteins, we conducted a comparative proteomic analysis. Microsomal proteins were isolated from roots of Arabidopsis seedlings left untreated or treated with 250 mM NaCl for 2 h, and resolved by 2D gel electrophoresis. In this study, we focus on root tissue for a number of reasons. The root is the site of salt uptake, and thus the physiology of its salt response has been well characterized (Davies and Zhang, 1991; Kiegle et al., 2000). Moreover, the root is almost devoid of ribulose 1,5-bisphosphate carboxylase/oxygenase (Rubisco), the most abundant leaf protein, which limits protein loading on 2D-gels and thus prevents visualization of low-abundance proteins.

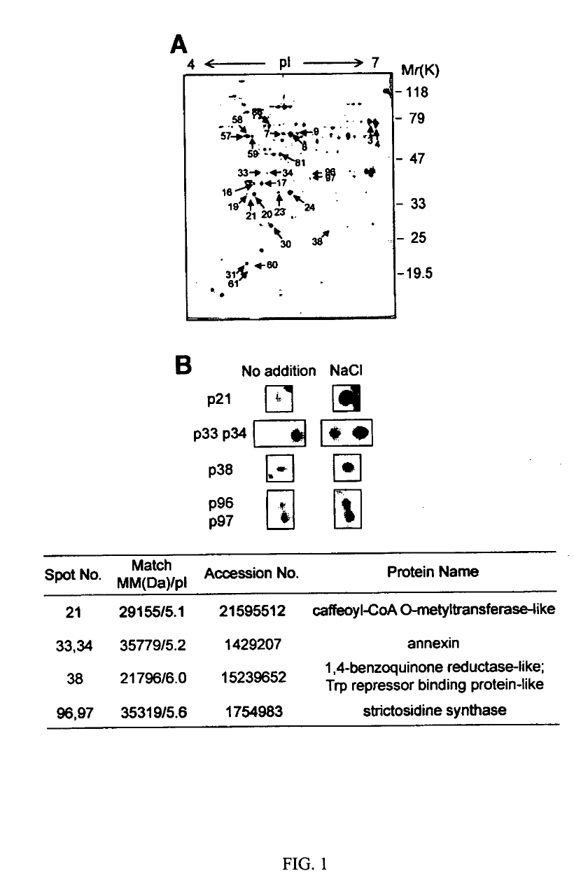

A 2D-gel of root microsomal proteins is shown in FIG. 1A. Protein spots were evenly distributed between pH 4-7 and molecular masses of 20-120 kDa. We reproducibly detected about 350 spots using image analysis software. In response to salt treatment, 34 proteins were up- or down-regulated with a greater than 2-fold change (data not shown). The example of changed spots is displayed in FIG. 1B.

Among the salt-responsive proteins, we initially selected p33 and p34 (spot # 33, 34) for further characterization. Both p33 and p34 represent the same protein, ANX1. p33 and p34 proteins migrated with a molecular mass of 40 kDa, which is slightly larger than the theoretical molecular size of ANX1 (36 kDa). The apparent pI values of p33 and p34 on a 2D-gel are consistent with the theoretical pI (5.19). Annexins are a multigene family of Ca²⁺-dependent membrane binding proteins that have been well characterized in animal cells (Gerke and Moss, 2002). The finding that ANX1 is present as two spots (p33 and p34) indicates posttranslational modification (e.g. phosphorylation) of the protein (FIG. 1B).

Expression of ANX1 in Tissues

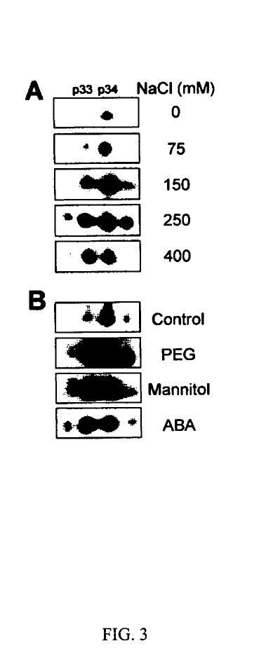

To further characterize ANX1, we generated an antibody against an ANX1-specific peptide (amino acids 204 to 215). The specificity of the anti-ANX1 antibody was examined by Western blot analysis. The antibody specifically recognized recombinant ANX1 protein generated in E. coli (data not shown). Moreover, both p33 and p34 protein spots were detected by the antibody on a 2D-gel prepared with plant extracts (FIG. 3).

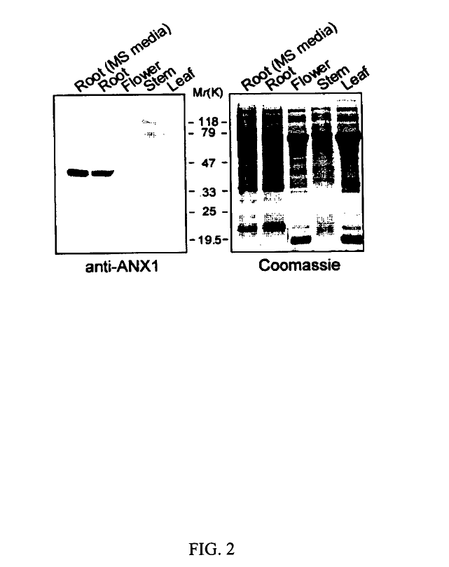

The expression pattern of ANX1 in tissues was determined by Western blot analysis. ANX1 was expressed predominantly in root, but not in flower, stem or leaf tissues (FIG. 2). The level of ANX1 in roots from Arabidopsis grown in soil was similar to that in Arabidopsis roots cultured in Murashige and Skoog (MS) media used throughout the experiments. Expression of ANX1 proteins in response to NaCl and other abiotic stress The salt response of ANX1 expression was further investigated by Western blotting. 2D-gels prepared with root microsomal proteins were probed with an anti-ANX1 antibody. In a dose-response experiment, ANX1 proteins were induced by treatment with NaCl at different concentrations. Proteins were most strongly induced at 250 mM NaCl (FIG. 3A).

Next we examined whether ANX1 expression is affected by ABA and other stress. We found that ANX1 proteins were induced by ABA (FIG. 3B). Treatment with mannitol and polyethylene glycol (PEG) additionally elevated ANX1 levels (FIG. 3B), suggesting that the protein is sensitive to ABA and general osmotic stress.

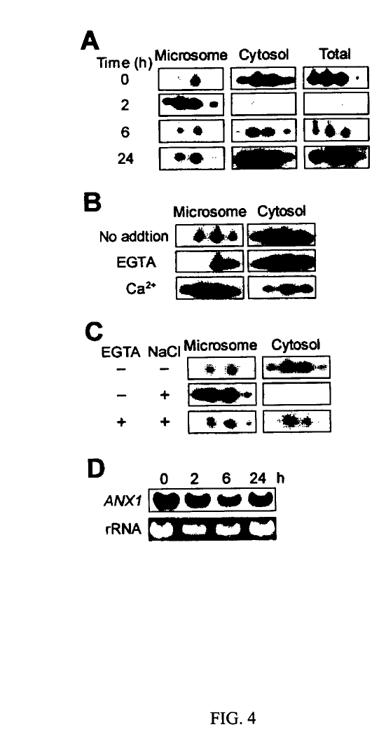

Immunoblotting of 2D-gels with an anti-ANX1 antibody revealed at least four spots, including p33 and p34 visualized by silver staining, demonstrating the increased sensitivity of immunoblotting (FIGS. 3 and 4). We propose that ANX1 undergoes posttranslational modifications, based on the array of horizontal spots observed on the 2D-gel, which is a typical pattern of posttranslationally modified proteins. Posttranslational changes in ANX1 proteins in response to salt stress The distribution of ANX1 proteins in cells was examined in the microsomal and cytosolic fractions from Arabidopsis roots grown under normal conditions. ANX1 was detected in both the cytosol and microsome, but was about 10-fold more abundant in the cytosol (FIGS. 4A and 4B). Whether microsomal ANX1 is distinct from the cytosolic form with respect to function and structure remains to be elucidated.

We compared the salt-induced changes in ANX1 proteins of the microsomal and cytosolic fractions by Western analysis of 2D-gels. ANX1 protein levels were considerably enhanced in the microsome following 2 h of salt treatment and fully recovered at 24 h (FIG. 4A). It was noticeable that the expression pattern was reversed in the cytosol, being almost completely abolished at 2 h of salt treatment and recovered thereafter. The pattern of expression of total ANX1 was similar to that of cytosolic ANX1, consistent with the finding that the cytosol constitutes the major fraction. The results imply that salt treatment affects ANX1 proteins in two ways, specifically, translocation from the cytosol to the membrane and protein turnover in the cytosol.

The Ca²⁺ dependency of the posttranslational changes in ANX1 proteins was examined. Plant extracts were incubated with either Ca²⁺ or EGTA before fractionation. While Ca²⁺ stimulated the movement of ANX1 proteins to the membrane, EGTA reversed the direction of translocation (FIG. 4B). We further investigated the Ca²⁺ effect on salt response of ANX1 proteins in vivo. The subcellular distribution of ANX1 proteins was determined in Ca²⁺-depleted plants incubated in MS media containing EGTA. Translocation to the membrane and degradation of ANX1 proteins were both inhibited (FIG. 4C). ANX1 degradation was only partially affected, which may be due to incomplete Ca²⁺ chelation in plants. The results strongly suggest that the salt response of ANX1 proteins is mediated by Ca²⁺.

We investigated whether the salt response of ANX1 proteins is observed at the transcript level. Northern blot analysis revealed that in contrast to the salt-induced changes in proteins, the transcript was not affected (FIG. 4D). The ANX1 level even slightly decreased over time. The data indicate that ANX1 is regulated, not by expression, but posttranslationally by the translocation and turnover of proteins.

T-DNA Insertion Mutants of ANX1 Genes

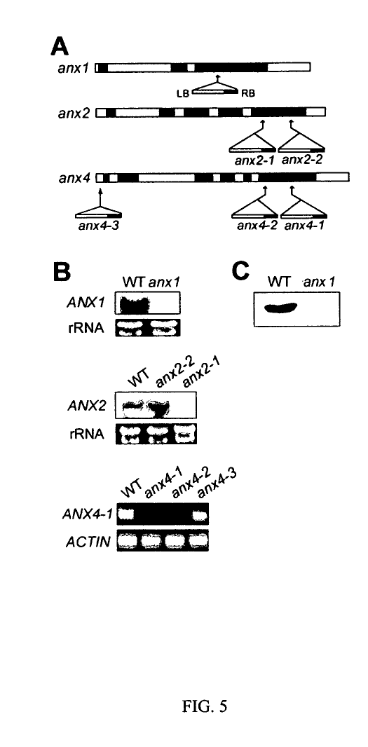

To determine the in vivo function of ANX1, we searched the Salk Institute insertion sequence database for anx1 T-DNA insertion mutants. We obtained an anx1 mutant as well as anx2 and anx4, other mutants of annexin family members. For anx2 and anx4, two and three different alleles were isolated, respectively (FIG. 5A). The anx1 mutant contains the T-DNA insert in the third exon, while the two anx2 alleles (anx2-1 and anx2-2) contain the insert in the fifth exon. In the three anx4 mutants, T-DNA is present in the sixth exon (anx4-1 and anx4-2) and in the 5′-untranslated region (UTR) (anx4-3). RNA analyses revealed that in some isolated mutants, the expression of each corresponding anx gene was almost completely suppressed compared to wild type (FIG. 5B), which was additionally verified by Western analysis in the case of anx1 (FIG. 5C). Accordingly, anx1, anx2-1 and anx4-1 were representatively selected for further experiments.

Sensitivity of anx T-DNA Insertion Mutants to NaCl

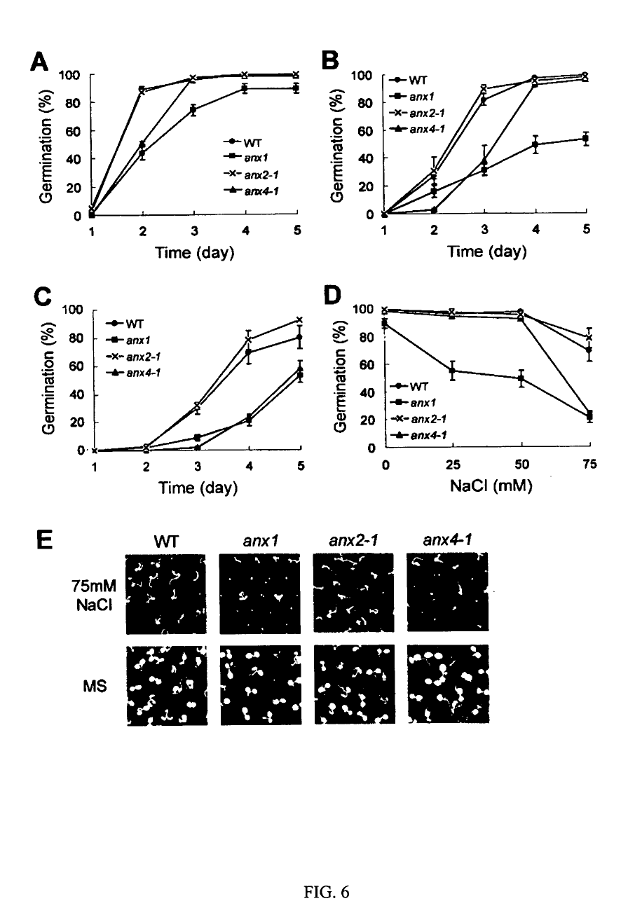

To assess the function of annexins in abiotic stress signaling, we determined the sensitivity of seed germination of anx mutants to NaCl. The anx1, anx2-1 and anx4-1 mutants were allowed to germinate in media containing various concentrations of NaCl. In MS media, anx1 displayed slightly decreased germination, with a rate of 85% (FIG. 6A). The anx2-1 and anx4-1 mutants germinated normally, similar to wild type. While only half the anx1 seeds germinated in the presence of 50 mM NaCl, anx4-1 was just delayed in the germination with levels comparable to wild type at 4 days after treatment (FIG. 6B). However, anx1 and anx4-1 mutant seeds displayed more severely defective germination at 75 mM NaCl than wild type and anx2-1 (FIG. 6C). The anx2-1 mutant displayed similar germination patterns to wild type at all concentrations of salt examined. ANX2 seems to play different roles in other than salt response, in contrast to ANX1 and ANX4. No differences were detected between anx4-1 and anx4-2 with respect to salt response (data not shown).

Although both anx1 and anx4-1 displayed hypersensitivity to NaCl, slightly different patterns were obtained in response to salt. Unlike anx1, anx4-1 displayed a sudden decrease in germination at 75 mM NaCl (FIG. 6D). As shown in FIG. 6E, seed germination of anx1 and anx4-1 was significantly affected (80% inhibition) in MS media containing 75 mM NaCl. The growth of germinated anx1 and anx4-1 plants was arrested after radicles emerged and resumed upon transfer to MS media (data not shown). The results collectively suggest that ANX1 and ANX4 are implicated in salt stress response in plants.

Sensitivity of anx1 and anx4 to General Osmotic Stress

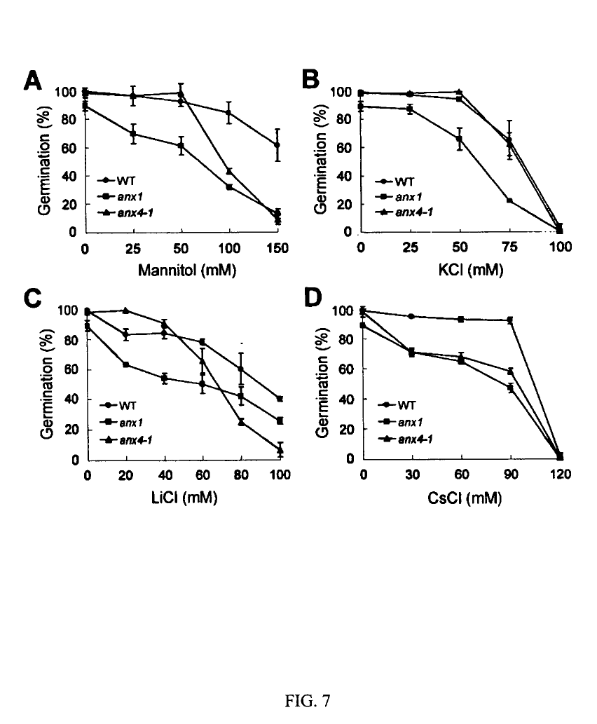

To determine whether the salt response of anx1 and anx4-1 results from an ionic or osmotic effect or both, germination was examined in the presence of several different ions, including KCl, LiCl and CsCl, and mannitol as an osmotic reagent. Both anx1 and anx4-1 plants were sensitive to mannitol, although anx4-1 was less sensitive, similar to data observed with NaCl (FIG. 7A). Interestingly, anx1 displayed defective germination in the presence of KCl and CsCl, but was less sensitive to LiCl. In contrast, anx4-1 was sensitive to LiCl and CsCl, but not KCl (FIGS. 7B, 7C and 7D). The results indicate that anx1 and anx4-1 are affected by general osmotic stress and partially in an ion-specific manner, as suggested by their differential ionic specificity.

Sensitivity of anx1 and anx4 to ABA

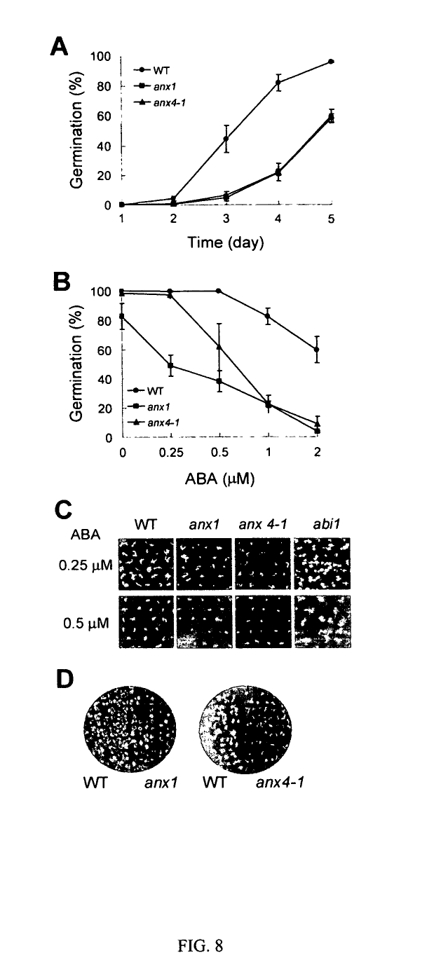

Earlier studies suggest that ABA mediates drought and salt stress response (Leung and Giraudat, 1998; Shinozaki and Yamagucho-Shinozaki, 2000). To test this, we investigated the germination of anx1 and anx4-1 mutant plants in media containing various concentrations of ABA. Both anx1 and anx4-1 exhibited defective germination in the presence of ABA (FIG. 8). In general, anx1 was more sensitive than anx4-1, particularly at lower concentrations of ABA (FIG. 8B). This result is consistent with that obtained from NaCl treatment (FIG. 6).

Germination of anx1 and anx4-1 plants is inhibited in media containing ABA compared to wild type and abi1, the ABA-insensitive mutant line (Koornneef et al., 1984) (FIG. 8C). Moreover, growth of anx1 and anx4-1 plants was impaired after radicles emerged, whereas wild-type plants continued to grow and get green (FIG. 8D). We also found that anx1 and anx4-1 were in a state of growth arrest in the presence of ABA and resumed normal growth upon transfer to ABA-deficient MS media, as observed with NaCl (data not shown).

Discussion

Identification of ANX1 in the Root Microsomal Proteome

Membrane proteins play important roles in various cellular processes, modulating diverse signaling pathways. Many signals are initially perceived and transduced through active molecules located in the membrane, which regulate cell-cell interactions and responses to the environment. Therefore, we targeted the microsomal proteome containing active proteins such as receptors, channels and membrane-associated signaling molecules for analysis. In this study, proteomic analyses led to the identification of the ANX1 protein. Levels of ANX1 increased upon NaCl treatment in the root microsomal proteome from Arabidopsis. Annexins are a family of Ca²⁺-dependent membrane binding proteins that exist in nearly all species, from fungi to human (Gerke and Moss, 2002). Annexins have been extensively studied in animal cells. These proteins are multifunctional and play important roles in various cellular processes, including membrane trafficking and organization, regulation of ion channel activity, phospholipid metabolism, inflammatory response and mitotic signaling (Raynal and Pollard, 1994).

Expression of ANX1

Although ANX1 is associated with the microsome, the protein is more abundant in the cytosol. The microsomal fraction comprises membranes originating from different organs, such as vacuole, chloroplast, Golgi and plasma membrane. Previous reports indicate that annexins are subcellularly localized in the plasma membrane, vacuole and nuclear periphery (Clark and Roux, 1995). To confirm subcellular localization, green fluorescent protein (GFP)-fused ANX1 was transiently expressed in BY-2 protoplasts. ANX1 was detected in both the cytosol and plasma membrane, and the GFP signal was significantly enhanced in the plasma membrane upon salt and ABA treatment (data not shown).

Posttranslational Changes in ANX1 Proteins

ANX1 RNA expression was not affected, but protein levels were significantly altered upon the addition of NaCl into the medium, implying that the protein is subjected to translational and/or posttranslational regulation. Within 2 h of salt treatment, ANX1 protein levels were considerably increased in the membrane and concurrently diminished in the cytosol. This salt-induced subcellular change was accompanied by a net decrease in total ANX1 proteins, which correlates with the finding that the major fraction of ANX1 exists in the cytosol. These results indicate that salt stress induces dynamic changes in ANX1 proteins, i.e. subcellular redistribution and turnover of existing proteins.

In many signaling processes, regulatory proteins are recruited from the cytosol to the membrane (Didichenko et al., 1996; Park et al., 2000; Oancea et al., 2003). Membrane association is often triggered by posttranslational modifications, such as phosphorylation, lipidation and glycosylation, and/or protein-protein interactions (Iwata et al., 1998). ANX1 was observed as at least four spots with different pls on a 2D-gel, suggesting posttranslational modifications. We are currently investigating the possibility of phosphorylation and other modifications of ANX1 proteins, as evidenced in animal cells (Gerke and Moss, 2002). Following stress treatment, ANX1 spots moved to the membrane together, implying that the modifications are not directly related to membrane association. ANX1 spots remaining in the cytosol were indistinguishable from those in the membrane, supporting this finding. It is assumed that ANX1 is recruited to the membrane through protein-protein interactions. Our preliminary data show that the sizes of the ANX1-associated complexes on a native gel are distinct between cytosolic and membrane fractions and altered before and after exposure of plants to stress stimuli (data not shown). Therefore, identification of the interacting components in ANX1 complexes should facilitate elucidation of the specific functions of the protein and the functional significance of membrane association in stress responses. With regard to protein turnover, proteolysis, particularly the ubiquitin/26S proteasome (Ub/26S) pathway, is one of the most important regulatory mechanisms controlling cellular functions in plants (Vierstra, 2003). Several known signaling components, including phyA, HY5/HYH, AUX/IAA, NACl, E2F and AB15, have been identified as target substrates. There is a previous report that annexins may be regulated by proteolysis, likely through lysosomal pathway, in rat lung tissue (Barnes and Gomes, 2002). Whether ANX1 is a selective target for Ub/26S or other proteolytic pathways would be an intriguing question.

Annexins are characterized by their ability to bind phospholipids in a Ca²⁺-dependent manner. In this study, we provide evidence that Ca²⁺ mediates the association of ANX1 proteins with the membrane. The inclusion of Ca²⁺ in plant extracts induced binding of ANX1 to the membrane, which was reversed by the addition of EGTA. In plants incubated in Ca²⁺-chelated media, ANX1 lost the ability to respond to salt stress, since both accumulation in the membrane and degradation in the cytosol were inhibited. These results imply that the salt stress-induced response of ANX1 is specifically regulated by Ca²⁺.

Functions of ANX1 and ANX4

In this study, we demonstrate that ANX1 is involved in the osmotic stress response. The anx1 mutant showed hypersensitivity to ABA and osmotic stress such as NaCl, LiCl, CsCl, KCl and mannitol in germination. In addition to anx1, anx4 was defective in germination under stress conditions. While anx1 and anx4 plants responded similarly to stress, they exhibited slightly different responses to various osmotic stress, with distinct ion selectivities and kinetics of germination. This may be due to differences in temporal and spatial expression and expression levels of proteins. ANX1 and ANX4 may have distinct, further defined roles in stress response in plants.

Therefore, we propose that ANX1 senses the Ca²⁺ signal elicited by ABA and stress, and transmits it to downstream signaling pathways via dual mechanisms of protein degradation and translocation to the membrane. Degradation may release the interacting molecules and translocation may enhance association with other molecules in the membrane, both activating the downstream signaling cascade. Receptors, channels and kinases are good candidates for interacting partners.

### REFERENCES

Barnes, J. A., and Gomes, A. V. (2002). Proteolytic signals in the primary structure of annexins. Mol. Cell. Biochem. 231, 1-7.
Clark, G. B., and Roux, S. J. (1995). Annexins of plant cells. Plant Physiol. 109, 1133-1139.
Davies, W. J., and Zhang, J. H. (1991). Root signals and the regulation of growth and development of plants in drying soil. Annu. Rev. Plant Physiol. Plant Mol. Biol. 42, 55-76.
Didichenko, S. A., Tilton, B., Hemmings, B. A., Ballmer-Hofer, K., and Thelen, M. (1996). Constitutive activation of protein kinase B and phosphorylation of p47phox by a membrane-targeted phosphoinositide 3-kinase. Curr. Biol. 6, 1271-1278.
Droillard, M., Boudsocq, M., Barbier-Brygoo, H., and Lauriere, C. (2002). Different protein kinase families are activated by osmotic stresses in Arabidopsis thaliana cell suspensions. Involvement of the MAP kinases AtMPK3 and AtMPK6. FEBS Lett. 527, 43-50.
Giraudat, J., Parcy, F., Bertauche, N., Gosti, F., and Leung, J. (1994). Current advances in abscisic acid action and signaling. Plant Mol. Biol. 26, 1557-1577.
Grill, E., and Himmelbach, A. (1998). ABA signalin transduction. Curr. Opin. Plant Biol. 1, 412-418.
Ichimura, K., Mizoguchi, T., Yoshida, R., Yuasa, T., and Shinozaki, K. (2000). Various abiotic stresses rapidly activate Arabidopsis MAP kinases ATMPK4 and ATMPK6. Plant J. 24, 655-665.
Iwata, S., Lee, J. W., Okada, K., Lee, J. K., Iwata, M., Rasmussen, B., Link, T. A., Ranaswamy, S., and Jap, B. K. (1998). Complete structure of the 11-subunit bovine mitochondrial cytochrome bc1 complex. Science 281, 64-71.
Jensen, O. N., Wilm, M., Shevchenko, A., and Mann, M. (1999). Sample preparation methods for mass spectrometric peptide mapping directly from 2-DE gels. Methods Mol. Biol. 112, 513-530.
Kiegle, E., Moore, C. A., Haseloff, J., Tester, M. A., and Knight, M. R. (2000). Cell-type-specific calcium responses to drought, salt and cold in the Arabidopsis root. Plant J. 23, 267-278.
Knight, H., and Knight, M. R. (2001). Abiotic stress signaling pathways: Specificity and cross-talk. Trends Plant Sci. 6, 262-267.
Knight, H., Trewavas, A. J., and Knight, M. R. (1996). Cold calcium signaling in Arabidopsis involves two cellular pools and a change in calcium signature after acclimation. Plant Cell 8, 489-503.
Knight, H., Trewavas, A. J., and Knight, M. R. (1997). Calcium signalling in Arabidopsis thaliana responding to drought and salinity. Plant J. 12, 1067-1078.
Koornneef, M., Reuling, G., and Karssen, C. (1984). The isolation and characterization of abscisic acid-insensitive mutants of Arabidopsis thaliana. Physiol. Plant. 61, 377-383.
Landry, F., Lombardo, C. R., and Smith, J. W. (2000). A method for application of samples to matrix-assisted laser desorption ionization time-of-flight targets that enhances peptide detection. Anal. Biochem. 279, 1-8.
Leung, J., and Giraudat, J. (1998). Abscisic acid signal transduction. Annu. Rev. Plant Physiol. Plant Mol. Biol. 49, 199-222.
Liu, J., and Zhu, J.-K. (1997). An Arabidopsis mutant that requires increased calcium for potassium nutrition and salt tolerance. Proc. Natl. Acad. Sci. USA 94, 14960-14964.
Liu, J., and Zhu, J.-K. (1998). A calcium sensor homolog required for plant salt tolerance. Science 280, 1943-1945.
Luan, S., Kudla, J., Rodriguez-Concepcion, M., Yalovsky, S., and Gruissem, W. (2002). Calmodulin and calcineurin B-like proteins: Calcium sensors for specific signal response coupling in plants. Plant Cell 14 (suppl.), S389-S400.
Murashige, T., and Skoog, F. (1962). A revised medium for rapid growth and bioassays with tobacco tissue culture. Physiol. Plant. 15, 473-497.
Oancea, E., Bezzerides, V. J., Greka, A., and Clapham, D. E. (2003). Mechanism of persistent protein kinase D1 translocation and activation. Dev. Cell 4, 561-574.
Park, J. Choi, H. J., Lee, S., Lee, T., Yang, Z., and Lee, Y. (2000). Rac-related GTP-binding protein in elicitor-induced reactive oxygen generation by suspension-cultured soybean cells. Plant Physiol. 124, 725-732.
Qiu, Q. S., Guo, Y., Dietrich, M. A., Schumaker, K. S., and Zhu, J.-K. (2002). Regulation of SOS1, a plasma membrane Na⁺/H⁺ exchanger in Arabidopsis thaliana, by SOS2 and SOS3. Proc. Natl. Acad. Sci. USA 99, 8436-8641.
Quintero, F. J., Ohta. M., Shi. H., Zhu. J. K., and Pardo, J. M. (2002). Reconstitution in yeast of the Arabidopsis SOS signaling pathway for Na⁺ homeostasis. Proc. Natl. Acad. Sci. USA 99, 9061-9016.
Raynal, P., and Pollarad, H. B. (1994). Annexins: the problem of assessing the biological role for gene family of multifunctional Ca²⁺-and phospholipid-binding proteins. Biochem. Biophys. Acta 1197, 63-93.
Romeis. T., Ludwig, A. A., Martin, R., and Jones, J. D. (2001). Calcium-dependent protein kinases play an essential role in a plant defence response. EMBO J. 20, 5556-5567.
Sajio, Y., Hata, S., Kyozuka, J., Shimamoto, K., and Izui, K. (2000). Over-expression of a single Ca²⁺-dependent protein kinase confers both cold and salt/drought tolerance on rice plants. Plant J. 23, 319-327.
Sheen, J. (1996). Ca²⁺ dependent protein kinases and stress signal transduction in plants. Science 274, 1900-1902.
Shinozaki, K., and Yamaguchi-Shinozaki, K. (2000). Molecular response to dehydration and low temperature: Differences and cross-talk between two stress signaling pathways. Curr. Opin. Plant Biol. 3, 217-223.
Skriver, K., and Mundy, J. (1990). Gene expression in response to abscisic and osmotic stress. Plant Cell 2, 503-512.
Townely, H. E., and Knight, M. R. (2002). Calmodulin as a potential negative regulator of Arabidopsis COR gene expression. Plant Physiol. 128, 1169-1172.
Trewavas, A. (1999). Le calcium, C'est la vie: calcium makes waves. Plant Physiol. 120, 1-6.
Vierstra, R. D. (2003). The ubiquitin/26S proteasome pathway, the complex last chapter in the life of many plant proteins. Trends Plant Sci. 8, 135-142.
Wu, S. J., Ding, L., and Zhu, J.-K. (1996). SOS1, a genetic locus essential for salt tolerance and potassium acquisition. Plant Cell 8, 617-627.
Xiong, L., Schumaker, K. S., and Zhu, J.-K. (2002). Cell signaling during cold, drought, and salt stress. Plant Cell 14 (suppl.), S165-S183.
Yamaguchi-Shinozaki, K., and Shinozaki, K. (1994). A novel cis-acting element in an Arabidopsis gene is involved in responsiveness to drought, low-temperature, or high-salt stress. Plant Cell 6, 251-264.
Zeevaart, J. A. D., and Creelman, R. A. (1998). Metabolism and physiology of abscisic acid. Annu. Rev. Plant Physiol. Plant Mol. Biol. 39, 439-473.
Zhu, J.-K. (2002). Salt and drought stress signal transduction in plants. Annu. Rev. Plant Biol. 53, 247-273.
Zhu, J.-K., Liu, J., and Xiong, L. (1998). Genetic analysis of salt tolerance in arabidopsis. Evidence for a critical role of potassium nutrition. Plant Cell 10, 1181-1191.
Zielinski, R. E. (1998). Calmodulin and calmodulin-binding proteins in plants. Annu. Rev. Plant Physiol. Plant Mol. 49, 697-725.

1. An isolated nucleic acid molecule encoding a protein with the activity of an annexin or encoding a fragment of such a protein with the biological activity, which is selected from the group consisting of:
(a) nucleic acid molecules encoding a polypeptide with the amino acid sequence of SEQ ID NO: 2;
(b) nucleic acid molecules encoding a polypeptide with the amino acid sequence of SEQ ID NO: 4;
(c) nucleic acid molecules comprising the coding region of the nucleotide sequence of SEQ ID NO: 1;
(d) nucleic acid molecules comprising the coding region of the nucleotide sequence of SEQ ID NO: 3;
(e) nucleic acid molecules that hybridize under stringent conditions to nucleic acid molecules of (a), (b), (c) or (d); and
(f) nucleic acid molecules that are degenerate to the nucleic acid molecules of any one of (a), (b), (c), (d) or (e).

2. The nucleic acid molecule according to claim 1, wherein the encoded protein is an annexin related to the osmotic stress and abscisic acid (ABA) signaling.

3. Nucleic acid molecules according to claim 1, wherein said nucleic acid is DNA.

4. Nucleic acid molecules according to claim 1, wherein said nucleic acid is RNA.

5. A vector containing a nucleic acid molecule according to claim 3.

6. The vectors according to claim 5, wherein the nucleic acid molecule is ligated to regulatory elements of promoters, terminators and signals for polyadenylation, for the expression of the annexin in prokaryotic or eukaryotic cells.

7. A host cell which is stably transformed with a vector according to claim 6.

8. The host cell according to claim 7, wherein the host cell is the prokaryotic, fungal, plant or animal cell.

9. A transgenic plant which is transformed by the nucleic acid of claim 1, wherein the reduction of the polypeptide with the amino acid sequence of SEQ ID NO: 2 or SEQ ID NO: 4 is achieved by an antisense.

10. A transgenic plant which is transformed by the nucleic acid of claim 1, wherein the increase of the polypeptide with the amino acid sequence of SEQ ID NO: 2 or SEQ ID NO: 4 is achieved by a sense.

11. A transgenic plant according to claim 9, wherein said transgenic plant displays a decreased resistance to environmental stress.

12. A transgenic plant according to claim 10, wherein said transgenic plant displays an increased resistance to environmental stress.
